(19) United States
(12) Patent Application Publication
(10) Pub. No.: US 2004/0220297 A1
(43) Pub. Date: Nov. 4, 2004

(54) Self curing cements

(76) Inventors: William Bonfield, Oat (GB); Julio San Roman Del Barrio, Las Matas (ES); Blanca Vazquez Lasa, Madrid (ES); Sanjukta Deb, West Sussex (GB)

Correspondence Address: BACON & THOMAS, PLLC, 625 SLATERS LANE, FOURTH FLOOR, ALEXANDRIA, VA, 22314

(21) Appl. No.: 10/148,177

(22) PCT Filed: Nov. 29, 2000
(86) PCT No.: PCT/GB00/04545

(30) Foreign Application Priority Data
Dec. 6, 1999 (GB) 9928837.5

(51) Int. Cl. A61K 6/08

(52) U.S. Cl. 523/116

(57) ABSTRACT

H₃C—(CH₂)ₐ—(CH(OH)CH₂)_{b}—(CH═CHCH₂)_{c}—(CH═CH(CH₂)₇)_{d}— (A)

A composition, for use in a dental or bone cement precursor composition, which comprises one or more compounds of general structure (I), wherein R₁ is H, methyl, ethyl, propyl or isopropyl, n=1, 2, 3 or 4, R₂ is a side chain having general structure (A), wherein a=4 to 16, b=0 or 1, c=0 or 1, d=0 or 1, and wherein the total number of carbon atoms in the side chain R₂ is not greater than 17 and one or more free radical activators.

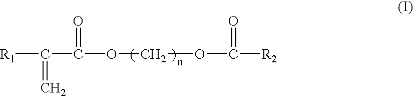

The present invention relates to compositions for use in cement precursor compositions and to novel polymeric self curing cements formed therefrom for use in dentistry and orthopaedic surgery. Polymeric bone cements have been used in orthopaedics for prostheses fixation for many years, the function of the bone cement being the immobilization of the prostheses. However, many short and long-term adverse effects have been reported due to the cement's chemical composition and/or physical properties.

Orthopaedic bone cements are predominantly based on poly(methylmethacrylate), PMMA. They are produced from self-curing bone cement precursor compositions which generally comprise two phases; a solid phase (usually a powder) and a liquid phase. The solid phase commonly comprises beads of prepolymerised PMMA or its copolymers together with one or more radical initiators and, optionally, one or more radiopaque agents. A radical initiator is a compound, such as a peroxide, which is capable of spontaneously forming free radicals. A radiopaque agent is a compound which is substantially opaque to radiation, in particular to radiation of the type used for medical diagnosis, such as X-rays. The liquid phase commonly comprises a polymerisable monomer, usually methylmethacrylate (MMA), together with one or more radical activators. A radical activator is a compound, such as a tertiary amine, which readily forms free radicals and thereby assists the propagation of free radical reactions. The liquid phase may also comprise one or more inhibitors and/or stabilisers which act to control the rate of free radical polymerisation. When the two phases of the bone cement precursor composition are mixed a polymerisation reaction results which generates a bone cement comprising particles of the solid powder embedded in an interstitial matrix of a newly formed polymer.

One disadvantage of bone cements based on PMMA is that the polymerisation reaction has a high exotherm and temperatures in the range of 70 to 110° C. may be generated at the centre of the cement mantle. The high rise in temperature is often a cause of necrosis and extensive bone damage has been reported to result from intramedullary cementation with PMMA based bone cements.

Another disadvantage results from the hypotensive effects of the monomer, methylmethacrylate, which may induce systemic effects if it enters the bloodstream. Unreacted monomer, present in the bone cement as a result of incomplete polymerisation of the bone cement precursor composition, can leach into the surrounding tissues leading to chemical necrosis.

The polymeric bone cement resulting from PMMA based bone cement precursor compositions is also a brittle material with low fracture toughness and poor fatigue characteristics. Improvements in the mechanical properties of acrylic bone cements have been achieved in a number of ways, for example by reinforcement of the cement, improving adhesion of the cement to bone, the production of lower modulus cements, the production of bioactive bone cements and the development of bone cement precursor composition dispensing and mixing techniques. Precursor composition mixing techniques are an important area of technology since they have a significant influence on the porosity of the resultant cements which ultimately influences their mechanical properties. Commercial cement manufacturers are increasingly using such techniques to improve the properties of bone cements. For a review of the state of the art reference is made to an article written by G. Lewis entitled “Properties of acrylic bone cement: State of the art review” (J. Biomed. Mater. Res (Appl. Biomaterials) 38, 155-182, 1997).

Asceptic loosening, that is loosening of the implant and fibrous membrane formed at the bone/cement interface with time, is another major problem associated with joint replacements. Fracture of bone cements and bone tissue necrosis are both believed to be major causes of asceptic loosening. As mentioned above, adverse biological responses to bone cements are mainly associated with low molecular weight residuals from the cement precursor composition, especially those having sufficient solubility in tissue fluids to be leached from the matrix into the surrounding tissues and the systemic circulation.

Another area of concern is in the common use of the tertiary amine activators N,N-dimethyl-p-toluidine (DMT) and N,N-dimethylaniline both of which belong to the N,N-dialkylaromatics, a class of compounds which are capable of reaction with DNA. Genotoxicity analysis has indicated that DMT in particular is able to induce chromosome alterations. Furthermore, the continued presence of DMT in cements which have been implanted for 2.5 to 10 years has been confirmed.

J. Biomed. Mater. Res., 1998, 43(2), 131-139 discloses the synthesis of 4-N,N-dimethylamine benzyl laurate and the use of this compound in the curing of acrylic cements at low temperatures. This activator was found to be less prone to leaching than he conventionally used activator, N,N-dimethyl-4-toluidene. J. Biomed. Mater. Res., 1999, Vol 48(J), 719-725 discloses further characteristics of an acrylic bone cement cured with the N,N-dimethylamino benzyl laurate activator.

In the light of the discussion above it is clear that there is a need to develop novel dental or bone cement precursor compositions which exhibit a lower exotherm on reaction and which produce novel bone cements with good mechanical properties and an improved biological response. It would also be advantageous to avoid the use of low molecular weight, water soluble chemicals and also potentially toxic chemicals such as DMT. Thus, investigations in this area have led to the identification of novel compounds which may usefully be employed in bone cement precursor compositions.

Accordingly, in one aspect the present invention provides a composition for use in a dental or bone cement precursor composition which comprises one or more compounds having the general structure shown in Formula (I) below:

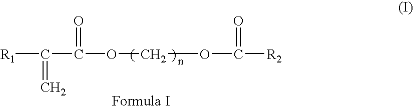

wherein:

R₁ is H, methyl, ethyl, propyl or isopropyl,

n=1, 2, 3 or 4,

R₂ is a side chain having the general structure shown below:

wherein:

a=4 to 16

b=0 or 1

c=0 or 1

d=0 or 1

and wherein the total number of carbon atoms in the side chain R₂ is not greater than 17 and one or more free radical activators.

In the compounds of formula (I), preferably R₁ is H or methyl, preferably n=2 and preferably the total number of carbon atoms in the side chain R₂ is in the range of from 7 to 17. More preferably the total number of carbon atoms in the side chain R₂ is in the range of from 9 to 17. Even more preferably the total number of carbon atoms in the side chain R₂ is in the range of from 11 to 17. Most preferably the total number of carbon atoms in the side chain R₂ is 11, 13, 15 or 17. It is preferable to use side chains which are longer because they will be expected to increase the hydrophobicity of the molecule thus lowering its water solubility. Longer side chains also produce a desirable plasticizing effect in the resultant bone cement.

H₃C—(CH₂)ₐ—(CH(OH)CH₂)_{b}—(CH═CHCH₂)_{c}—(CH═CH(CH₂)₇)_{d}—

Preferably, R₂ is a side chain of a fatty acid. Fatty acids are a well known class of chemical compounds. They are carboxylic acids derived from or contained in an animal or vegetable fat or oil. All fatty acids possess a linear side-chain which may consist of from 3 to 21 carbon atoms attached to the terminal —COOH group. The linear side-chains of fatty acids may be saturated or unsaturated and they most commonly possess an odd number of carbon atoms so that the fatty acid as a whole (including the —COOH group) possesses an even number of carbon atoms. More preferably, R₂ is a side chain selected from those found on the following fatty acids:

lauric acid, R₂=CH₃(CH₂)₁₀

palmitic acid, R₂=CH₃(CH₂)₁₄

stearic acid, R₂CH₃(CH₂)₁₆

oleic acid, R₂=CH₃(CH₂)₇CH═CH(CH₂)₇

ricinoleic acid, R₂=CH₃(CH₂)₅CH(OH)CH₂CH═CH(CH₂)₇

linoleic acid, R₂=CH₃(CH₂)₄CH═CHCH₂CH═CH(CH₂)₇

Preferably, the side-chain R₂ is unsaturated. This means that the polymerisable monomer is also able to undergo crosslinking. It is well known that a large number of physical properties such as modulus of elasticity, heat distortion, shrinkage and glass transition temperature, improve with crosslinking.

Preferably the dental or bone cement precursor composition comprises one or more of the compounds of Formula 1 (I) as defined above in an amount of from 2 to 30 wt %, more preferably from 5 to 15 wt %, based on the total weight of the composition.

The composition may comprise MMA in an amount of from 70 to 98 wt %, more preferably from 85 to 95 wt %, based on the total weight of the composition.

The composition may comprise one or more tertiary amine free radical activators in an amount of up to 7 wt %, preferably in an amount of from 5 to 7 wt %, based on the total weight of the composition, and/or one or more inhibitors in an amount of up to 0.01 wt %, preferably in an amount of from 0.0075 to 0.01 wt %, and/or one or more stabilisers in an amount of up to 0.01 wt %, preferably in an amount of from 0.0075 to 0.01 wt %.

Suitable tertiary amine activators are N,N-dimethyl-p-toluidine, dimethylamino ethylmethacrylate, N,N-dimethylaniline and compounds of Formula (II) below. Suitable inhibitors are quinone based compounds. Suitable stabilisers are quinone based compounds, such as hydroquinone.

The present invention also provides a composition, for use as a dental or bone cement precursor composition, which comprises a solid phase and a liquid phase, wherein the liquid phase comprises the first bone cement precursor composition described above.

The solid phase may comprise beads of prepolymerised MMA or copolymers of MMA with other monomers such as, for example, methacrylate. The beads may be present in an amount of from 70 to 95 wt %, more preferably from 80 to 95 wt %, based on the total weight of the solid phase.

The solid phase may also comprise one or more initiators in an amount of up to 3 wt %, preferably in an amount of from 2 to 3 wt %, and/or one or more radiopaque agents in an amount of up to 20 wt %, preferably in an amount of from 10 to 20 wt %, more preferably in an amount of from 15 to 20 wt % based on the total weight of the solid phase. Suitable initiators include, for example benzoyl peroxide. Suitable radiopaque agents may be selected from zirconia or barium sulphate.

The present invention also provides in a further aspect compounds for use as free radical activators in bone cement precursor compositions having the general structure shown in Formula (II) below:

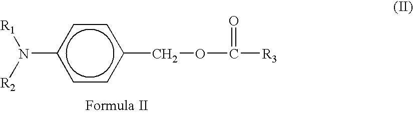

wherein:

R₁ is methyl, ethyl, propyl, isopropyl, butyl, isobutyl or tert-butyl,

R₂ is methyl, ethyl, propyl, isopropyl, butyl, isobutyl or tert-butyl,

R₃ is a side chain having the general structure shown below:

H₃C—(CH₂)ₐ—(CH(OH)CH₂_{b}—(CH═CHCH₂)_{c}—(CH═CH(CH₂)₇)_{d}

wherein:

a=4 to 16

b=0 or 1

c=0 or 1

d=0 or 1

and wherein the total number of carbon atoms in the side chain R₃ is not greater than 17, provided that the said compound is not 4-N,N-dimethylamino benzyl laurate (DML).

The advantages of the compounds of Formula II used as free radical activators in bone cement precursor compositions, as compared with DML, is that these cements have lower peak temperatures and shorter setting times. Furthermore, these cements have improved mechanical properties as compared with those prepared using DML.

Preferably in the compounds of Formula (II) R₁ and R₂ are methyl, and preferably the total number of carbon atoms in the side chain R₃ is in the range of from 7 to 17. More preferably the total number of carbon atoms in the side chain R₃ is in the range of from 9 to 17. Even more preferably the total number of carbon atoms in the side chain R₃ is in the range of from 11 to 17. Most preferably the total number of carbon atoms in the side chain R₃ is 11, 13, 15 or 17. It is preferable to use side chains which are longer because they will be expected to increase the hydrophobicity of the molecule thus lowering its water uptake. Longer side chains also produce a desirable plasticizing effect in the resultant bone cement.

Preferably, R₃ is a side chain of a fatty acid. More preferably, R₃ is a side chain selected from those found on the following fatty acids:

lauric acid, R₃=CH₃(CH₂)₁₀

palmitic acid, R₃=CH₃(CH₂)₁₄

stearic acid, R₃=CH₃(CH₂)₁₆

oleic acid, R₃=CH₃(CH₂)₇CH═CH(CH₂)₇

ricinoleic acid, R₃=CH₃(CH₂)₅CH(OH)CH₂CH═CH(CH₂)₇

linoleic acid, R₃=CH₃(CH₂)₄CH_{50 CHCH}₂CH═CH(CH₂)₇

The present invention also provides in a still further aspect a second composition for use as the liquid phase in a dental or bone cement precursor composition, the composition comprising one or more compounds of Formula (II) above and one or more polymerisable monomers.

Preferably the composition comprises one or more compounds of Formula (II) above in an amount of up to 7 wt %, more preferably in the range of from 3 to 7 wt %, based on the total weight of the composition.

The composition may comprise MMA as the polymerisable monomer. Preferably the MMA is present in an amount of from 70 to 95 wt %, more preferably from 85 to 90 wt %, based on the total weight of the composition. The composition may comprise one or more compounds of Formula (I) as the polymerisable monomer. Preferably compounds of Formula (I) are present in an amount of from 2 to 30 wt %, more preferably from 5 to 15 wt %, based on the total weight of the composition.

The composition may also comprise one or more inhibitors in an amount of up to 0.01 wt %, preferably in an amount of from 0.0075 to 0.01 wt % and/or one or more stabilisers in an amount of up to 0.01 wt %, preferably in an amount of from 0.0075 to 0.01 wt %.

Suitable inhibitors are quinone based compounds. Suitable stabilisers are quinone based compounds such as hydroquinone.

The present invention also provides a composition, for use as a dental or bone cement precursor composition, which comprises a solid phase and a liquid phase, wherein the liquid phase comprises the second composition described above.

The solid phase may comprise beads of prepolymerised MMA or copolymers of MMA with other monomers such as, for example, methacrylate. The beads may be present in an amount of from 70 to 95 wt %, more preferably from 80 to 95 wt %, based on the total weight of the solid phase.

The solid phase may also comprise one or more initiators in an amount of up to 3 wt %, preferably in an amount of from 2 to 3 wt %, and/or one or more radiopaque agents in an amount of up to 20 wt %, preferably in an amount of from 10 to 20 wt %, more preferably in an amount of 15 to 20 wt %, based on the total weight of the solid phase. Suitable initiators include, for example, benzoyl peroxide. Suitable radiopaque agents may be selected from zirconia or barium sulphate.

The present invention also encompasses a cement which has been produced by curing any one of the compositions described above as being suitable for use as a dental or bone cement precursor composition. These cements possess several advantages over those of the prior art when used as dental or bone cements and these advantages are described below.

The compounds of general formulae (I) and (II) possess a long aliphatic chain and are therefore hydrophobic. This increased hydrophobicity in comparison with, for example, methylmethacrylate results in a lower uptake in aqueous solutions and therefore a decrease in necrosis resulting from leaching of these compounds from the dental or bone cement into the surrounding tissues and systemic circulation. The compounds of Formulae (I) and (II) are also found to produce a plasticizing effect in the final dental or bone cement. This means that the material will be less brittle. The long chains of these compounds enable the polymeric chains to undergo deformation before breaking thus increasing the resistance of the material to crack propagation and increasing fatigue strength as well as providing additional means for the dissipation of biomechanical stress.

Furthermore, dental or bone cement precursor compositions comprising, in the liquid phase, a compound of Formula (II) as an activator and/or a compound of Formula (I) as a polymerisable monomer have been found to cure at lower temperatures than compositions known in the art. This would potentially reduce damage to tissues during formation of dental or bone cements in situ.

In a further embodiment the present invention also provides a process for forming dental or bone cements which comprises providing a solid phase and a liquid phase, mixing the two phases together and allowing polymerisation to occur, wherein the solid phase comprises polymer beads and one or more radical initiators and the liquid phase is selected from the first or second composition described above.

The present invention will be further described with reference to the following examples.

### EXAMPLE 1

Synthesis of Ethylene Glycol Oleate Methacrylate (OMA)

2-hydroxyethyl methacrylate was introduced into a three-necked flask and dissolved in solvent (diethyl ether) at room temperature. An equimolar amount of triethylamine was added to the reaction mixture. An equimolar amount of oleoyl chloride was added under constant stirring and the reaction was allowed to proceed for 48 hours at room temperature. The reaction mixture was filtered to separate the amine chlorhydrate formed and the solution was concentrated under reduced pressure to yield the diester OMA (shown below) with a yield of 70% with respect to 2-hydroxyethyl methacrylate. The OMA was characterised by ¹H-NMR spectroscopy using deuterated chloroform (5% wt/v) as solvent and tetramethylsilane (TMS) as internal standard: δ_{H} 6.11 and 5.56 (2H, CH₂═C(CH₃)CO), 1.92 (3H, CH₂═C(CH₃)CO), 4.31 (2H, O(CH₂)₂O), 2.30 (2H, COCH₂CH₂), 1.59 (2H, COCH₂CH₂), 1.98 (4H, CH₂CH═CHCH₂), 5.32 (2H, CH₂CH═CHCH₂), 1.04 to 1.45 (20H, (CH₂)₄ and (CH₂)₆CH₃), 0.85 (3H, (CH₂)₆CH₃). The purity of the product was greater than 98%.

CH₂═C(CH₃)CO—O(CH₂)₂O—COCH₂CH₂(CH₂)₄CH₂CH═CH—CH₂(CH₂)₆CH₃

OMA

### EXAMPLE 2

Synthesis of 4-N,N-dimethylaminobenzyloleate (DMAO)

Equimolar amounts of 4-N,N-dimethylaminobenzyl alcohol and triethylamine were dissolved in solvent (diethyl ether) at room temperature. An equimolar quantity of oleoyl chloride was added under constant stirring and the reaction was allowed to proceed for 48 hours at room temperature. The reaction medium was filtered to separate the amine chlorhydrate formed and the solution was concentrated under reduced pressure to yield DMAO (shown below) with a yield of 70% with respect to 4-N,N-dimethylaminobenzyl alcohol. The DMAO was characterised by ¹H-NMR spectroscopy using deuterated chloroform (5% wt/v) as solvent and tetramethylsilane (TMS) as internal standard: δ_{H} 2.94 (6H, (CH₃)₂N), 7.22 and 6.69 (4H, C₆H₄CH₂O), 4.99 (2H, C₆H₄CH₂O), 2.28 (2H, COCH₂CH₂), 1.60 (2H, COCH₂CH₂), 1.98 (4H, CH₂CH═CHCH₂), 5.32 (2H, CH₂CH═CHCH₂), 1.15 to 1.43 (20H, (CH₂)₄ and (CH₂)₆CH₃), 0.86 (3H, (CH₂)₆CH₃). The purity of the product was greater than 98%.

(CH₃)₂N—C₆H₄CH₂O—COCH₂CH₂(CH₂)₄CH₂CH═CH—CH₂(CH₂)₆CH₃

DMOA

### COMPARATIVE EXAMPLE I AND EXAMPLES I TO IV

Formulation of Bone Cements using DMAO as Activator

Bone cement precursor compositions were formulated with and without the activator compound DMAO which was synthesised as in Example 2 above. The compositions are given in Table 1 below:

The following abbreviations are used in the tables:

P(MA/MMA)=Poly(methylacrylate-methylmethacrylate) copolymer. The ratio following the abbreviation indicates the relative amounts of methylacrylate to methylmethacrylate in the copolymer

BPO=Benzoyl peroxide

MMA=Methylmethacrylate

DMT=N,N-dimethyl-4-toluidine

DMAO=4-N,N-dimethylaminobenzyloleate

OMA=Ethylene glycol methacrylate oleate

SD=Standard Deviation

“QL beads” are poly(methylmethacrylate) beads having the morphological characteristics given below. They are commercially available from Industrial Quirurgicas de Levante:

Average Diameter, D=33.1 μm

Interval of D=10-60 μm

Molecular Number Average, Mₙ=97×10³

Glass Transition Temperature, T_{g}=103° C.

The composition of Comparative Example I is that of Palacos™ which is a commercially available bone cement precursor composition.

TABLE 1

| | | | Composition of | |
|---|---|---|---|---|
| | Composition of solid | | liquid phase | |
| Example | phase (wt %) | | (wt %) | |
| Comparative | P (MA/MMA) 6:94 | (70.00) | MMA | (97.98) |
| Example I | P (MA/MMA) 42:58 | (14.25) | DMT | (2.02) |
| | ZrO₂ | (15.00) | | |
| | BPO | (0.75) | | |
| Example I | P (MA/MMA) 6:94 | (70.00) | MMA | (94.16) |
| | P (MA/MMA) 42:58 | (14.25) | DMAO | (5.84) |
| | ZrO₂ | (15.00) | | |
| | BPO | (0.75) | | |
| Example | QL beads | (84.11) | MMA | (94.16) |
| II | ZrO₂ | (15.0) | DMAO | (5.84) |
| | BPO | (0.89) | | |
| Example | QL beads | (83.50) | MMA | (94.16) |
| III | ZrO₂ | (15.00) | DMAO | (5.84) |
| | BPO | (1.50) | | |
| Example | QL beads | (83.5) | MMA | (97.43) |
| IV | ZrO₂ | (15.00) | DMAO | (2.57) |
| | BPO | (1.50) | | |

For each example a total of 40 g of the solid phase and 20 ml of the liquid phase made up the final composition. These bone cement precursor compositions were mixed together to form cement doughs which cured to provide bone cements. A comparison of the curing parameters for the compositions is given in Table 2 below.

The peak temperature (Tₚₑₐₖ) is defined as the maximum temperature reached during the polymerisation reaction and it was recorded according to ASTM standard (F451). The two components of the bone cement precursor composition were mixed and some of the resulting dough was packed into the plunger cavity of a mould. A thermocouple was positioned within its junction in the centre of the mould at a height of 3 mm in the internal cavity. The plunger subsequently was seated on the filled mould cavity and tightened with a G-clamp. Time was measured from onset of mixing the solid and liquid phases and the temperature was recorded. An average of two measurements was conducted as per the standard. Exotherms were registered at 25° C.

The dough time (t_{dough}) represents the time at which the polymerising mass does not adhere to a surgical glove. This is the time at which the cement can be implanted in the body, for example in the femoral cavity.

The setting time (t_{setting}) was determined according to ASTM standard (F451) as the time when the temperature of the polymerising mass is as follows:

T_{amb}+(Tₘₐₓ−T_{amb)/}2

where Tₘₐₓ is the maximum temperature in ° C. and T_{amb} is the ambient temperature, 23° C.

Residual monomer content was determined by ¹H-NMR spectroscopy. Samples were stored in air at room temperature for a week before being analysed. Three samples of each type were dissolved in deuterated chloroform and the spectrum recorded on a Bruker 250 MHz spectrometer.

TABLE 2

| | | Residual | t_{dough} | t_{setting} | Tₚₑₐₖ |
|---|---|---|---|---|---|
| | | monomer | (min) | (min) | (° C.) |
| Example | Activator | (%) [SD] | [SD] | [SD] | [SD] |
| Comparative | DMT | 3.60 | 2.0 | 11 | 73 |
| Example I | | [0.19] | [0.12] | [0.27] | [0.98] |
| Example I | DMAO | 3.70 | 2.0 | 18 | 53 |
| | | [0.05] | [0.20] | [0.02] | [0.20] |
| Example | DMAO | 4.40 | 5.5 | 17 | 54 |
| II | | [0.59] | [0.20] | [0.04] | [0.15] |
| Example | DMAO | 3.50 | 4.5 | 16 | 62 |
| III | | [0.40] | [0.30] | [0.17] | [0.40] |
| Example | DMAO | 3.50 | 3.0 | 17 | 57 |
| IV | | [0.30] | [0.25] | [0.10] | [0.49] |

The mechanical properties for the bone cements resulting from the cured compositions are given in Table 3 below.

Tensile tests were performed on an Instron Universal testing machine with a cell load of 100KN and at a crosshead speed of 5 mm/min. An extensometer was used to measure displacement. Specimens were prepared by placing the cement dough in PTFE moulds and subsequently under a pressure of 1.4 MPa for approximately 20 min. The specimens were then stored under dry conditions for one week prior to testing. Dumbell specimens were made in accordance with ISO-527, and the average cross-section of the specimens was 5.0 mm×4.0 mm. A minimum of six specimens was tested for each batch.

TABLE 3

| | | Ultimate | | |
|---|---|---|---|---|
| | Compressive | Tensile | Young's | Strain to |
| | Strength | Strength | Modulus | Failure |
| | (MPa) | (MPa) | (MPa) | (%) |
| Example | [SD] | [SD] | [SD] | [SD] |
| Comparative | 81.1 | 42.1 | 3.58 | 2.6 |
| Example I | [3.67] | [3.06] | [0.3] | [0.59] |
| Example | 85.9 | 42.6 | 1.6 | 5.4 |
| II | [3.36] | [2.46] | [0.06] | [0.37] |
| Example | 85.6 | 41.2 | 1.54 | 5.0 |
| III | [2.18] | [2.27] | [0.14] | [0.27] |
| Example | 78.1 | 41.9 | 1.35 | 5.0 |
| IV | [2.95] | [1.70] | [0.07] | [0.12] |

The results of tensile and compressive tests of cements prepared with the activator DMAO and stored in saline solution (0.9%) for 5 weeks are shown in Table 4 below. Compressive strengths of the experimental cements were significantly (p<0.001) higher than the control except for the cement containing the lower concentration of DMAO (Example IV). A comparison of the ultimate tensile strengths showed that the incorporation of the amine DMAO in Comparative Example I and in the cement of Example II did not produce any statistically significant difference (p<0.001). Young's modulus showed a significant decrease (p<0.001) in comparison to that of Comparative Example 1 and the strain to failure values were not statistically significant.

TABLE 4

| | Compressive | Ultimate | Young's | Strain to |
|---|---|---|---|---|
| | Strength | Tensile | Modulus | Failure |
| | (MPa) | Strength | (GPa) | (%) |
| Example | [SD] | (MPa) [SD] | [SD] | [SD] |
| Comparative | 71.4 | 37.9 | 2.4 | 5.2 |
| Example I | [1.0] | [2.48] | [0.06] | [0.34] |
| Example I | 76.3 | 36.3 | 1.20 | 6.3 |
| | [2.6] | [1.07] | [0.05] | [0.24] |
| Example | 73.0 | 37.9 | 1.20 | 5.6 |
| II | [3.0] | [0.07] | [0.09] | [0.55] |
| Example | 75.6 | 37.8 | 1.26 | 5.3 |
| III | [2.6] | [1.98] | [0.06] | [0.17] |
| Example | 71.8 | 39.0 | 1.48 | 5.4 |
| IV | [2.9] | [1.26] | [0.14] | [0.40] |

### EXAMPLES V TO XI

Formation of Bone Cements using OMA

Bone cement precursor compositions were formulated with the monomer compound OMA synthesised in Example 1 above partly substituting MMA as the polymerisable monomer in the liquid phase. The compositions also included in the liquid phase the activator compound synthesised in Example 2 above. The compositions are given in Table 5 below. The ratio Solid:Liquid represents the ratio of the weight of the solid phase to the weight of the liquid phase in the final bone cement precursor composition.

TABLE 5

| | | | Composition of | | |
|---|---|---|---|---|---|
| | Composition of | | liquid phase | | Solid: |
| Example | solid phase (wt %) | | (wt %) | | Liquid |
| Example V | QL Beads | (98.00) | MMA | (84.16) | 1.8:1 |
| | BPO | (2.00) | OMA | (10.00) | |
| | | | DMAO | (5.84) | |
| Example | QL Beads | (98.00) | MMA | (79.16) | 1.8:1 |
| VI | BPO | (2.00) | OMA | (15.00) | |
| | | | DMAO | (5.84) | |
| Example | QL Beads | (98.00) | MMA | (89.16) | 1.5:1 |
| VII | BPO | (2.00) | OMA | (5.00) | |
| | | | DMAO | (5.84) | |
| Example | QL Beads | (98.00) | MMA | (84.16) | 1.5:1 |
| VIII | BPO | (2.00) | OMA | (10.00) | |
| | | | DMAO | (5.84) | |
| Example | QL Beads | (98.00) | MMA | (79.16) | 1.5:1 |
| IX | BPO | (2.00) | OMA | (15.00) | |
| | | | DMAO | (5.84) | |
| Example X | QL Beads | (88.00) | MMA | (84.16) | 1.8:1 |
| | BPO | (2.00) | OMA | (10.00) | |
| | ZrO₂ | (10.00) | DMAO | (5.84) | |
| Example | QL Beads | (88.00) | MMA | (84.16) | 1.5:1 |
| XI | BPO | (2.00) | OMA | (10.00) | |
| | ZrO₂ | (10.00) | DMAO | (5.84) | |

These bone cement precursor compositions were mixed together to form cement doughs which cured to provide bone cements. A comparison of the curing parameters for the compositions is given in Table 6 below.

The working time (t_{working}) is the time which a clinician has to manipulate and insert the cement into place. It is approximately equal to the time difference between the dough time (t_{dough}) and the setting time (t_{setting})

TABLE 6

| | OMA | t_{dough} | t_{setting} | t_{working} | Tₚₑₐₖ |
|---|---|---|---|---|---|
| Example | (wt %) | (min) | (min) | (min) | (min) |
| Example V | 10 | 7.00 | 19.0 | 10.25 | 61 |
| Example | 15 | 3.50 | 18.5 | 15.0 | 53 |
| VI | | | | | |
| Example | 5 | 10.50 | 21.0 | 10.5 | 64 |
| VII | | | | | |
| Example | 10 | 10.25 | 23.0 | 12.0 | 60 |
| VIII | | | | | |
| Example | 15 | 8.00 | 22.0 | 14.0 | 55 |
| IX | | | | | |
| Example X | 10 | 8.00 | 18.0 | 10.0 | 59 |
| Example | 10 | 15.00 | 27.0 | 12.0 | 59 |
| XI | | | | | |

The mechanical properties of the cements of Examples V to XI are compared with Comparative Example I and the results are given in Table 7 below.

TABLE 7

| | Ultimate Tensile | Young's | Strain |
|---|---|---|---|
| | Strength | Modulus | to Failure |
| | (MPa) | (Gpa) | % |
| EXAMPLE | [SD] | [SD] | [SD] |
| Comparative | 42.1 | 3.58 | 2.6 |
| Example I | [3.06] | [0.3] | [0.59] |
| Example V | 39.37 | 3.13 | 2.17 |
| | [2.33] | [0.41] | [0.43] |
| Example VI | 34.91 | 2.69 | 1.96 |
| | [1.16] | [0.47] | [0.77] |
| Example VII | 48.7 | 3.65 | 2.06 |
| | [2.20] | [0.90] | [0.10] |
| Example IX | 34.0 | 3.56 | 1.92 |
| | [5.30] | [0.66] | [0.34] |
| Example X | 37.47 | 3.26 | 2.44 |
| | [1.49] | [0.60] | [0.80] |
| Example XI | 34.91 | 2.69 | 1.96 |
| | [1.16] | [0.47] | [0.77] |

The results show that low amounts of a compound of Formula (I) incorporated into the bone cements provide good tensile strength and Young's Modulus, without impairing the elongation to failure.

From Table 2 it can be seen that the novel activator DMAO was effective in curing both commercial and experimental cements. The curing parameters with DMAO exhibited lower peak temperatures albeit with longer setting times. The setting times were nevertheless within the limits set by ISO standards. The residual monomer content was in the same range as for the commercial composition. Thus the activator DMAO was found to be effective in initiating the free radical polymerisation. The curing parameters and residual monomer content were indicative of effective polymerisation with a net lowering in peak temperature.

The mechanical properties shown in Table 3 indicate that bone cements formulated with the activator of the present invention exhibited comparable tensile strengths to that of Comparative Example 1. However, the mean values of compressive strengths were found to be statistically significantly different at levels (p<0.001) and a pairwise comparison using a Student Newman Keuls test indicated that the cement of Example II had a greater compressive strength than that of Comparative Example 1. The differences in the mean values of the Young's modulus showed that there was a significant decrease (p<0.001) in the cements of the invention in comparison to that of Comparative Example 1 and the strain to failure exhibited a significant increase (p<0.001) in comparison to that of Comparative Example 1. It appears that the small amount of the long chain activator behaves as a plasticizer. A further advantage of DMAO is that cytotoxicity tests show it to be non-toxic, in contrast with presently used activators such as DMT.

From Table 6 it can be seen that the combination of the novel activator DMAO with a monomer composition wherein some of the MMA is replaced with OMA was also effective in curing experimental cements. The curing parameters with DMAO and OMA exhibited lower peak temperatures and longer working times. The longer working time gives the clinician more time for manipulation and insertion of the cement. The replacement of some MMA with OMA should decrease the likelihood of unreacted monomer entering the systemic circulation.

With reference to Table 7, the mechanical properties of the cement containing 5% OMA (Example VII), exhibited superior mechanical properties in comparison to Comparative Example 1 and the other cements as shown in Table 6. Higher concentrations of the OMA monomer decreased the tensile strength, the decrease being statistically significant at p<0.001. Young's modulus for the cement of Example VII was not statistically different from that of the control and the same was obtained for the strain to failure, which means that low concentrations of the OMA monomer enhanced the tensile strength without impairing the other two parameters.

Glass transition temperatures, T_{g}, of the cements were determined by Differential Scanning Calorimetry (DSC) using a Perkin Elmer DSC7 interfaced to a thermal analysis data system TAC 7/DX. The dry samples were prepared in the form of thin films placed in aluminium pans and heated from 30 to 150° C. at a constant rate of 10° C./min. T_{g} was taken as the midpoint of the heat capacity transition.

T_{g} values of cements formulated with the activator DMAO were in the range 93-95° C. that is 20° C. lower than the corresponding cement cured with DMT (T_{g}=111° C.) indicating the elasticising effect produced by the presence of the oleic chain of the activator. Comparative Example 1 presented a value of T_{g} of 93° C. due to the presence of methyl acrylate units in the prepolymerised beads. T_{g} of cements formulated with DMAO and OMA were around 85° C. that is around 10° C. lower than that of Comparative Example 1 as a consequence of the increase in the oleic residues from both activator and monomer, although this value is high enough to avoid the risk of sinking of the prostheses in vivo conditions caused by creep, considering that under extreme conditions the T_{g} of the cement can fall approximately 20° C.

The values of glass transition temperature, number and weight average molecular weights and polydispersity of cements formulated with OMA are given in Table 8 below.

Molecular weight distributions were determined by Size Exclusion Chromatography (SEC)(Waters 510 with a refractive index detector series 200). A set of 10⁴ Å, 10³ Å and 500 Å PL-gel columns conditioned at 25° C. were used to elute the sample of 10 mg/ml concentration at 1 ml/min HPLC-grade chloroform flow rate. Calibration of SEC was carried out with monodiperse standard polystyrene samples obtained from Polymer Laboratories.

TABLE 8

| EXAMPLE | T_{g} (° C.) | Mₙ × 10⁵ | M_{w} × 10⁵ | Polydispersity |
|---|---|---|---|---|
| Example V | 85 | 1.3 | 3.4 | 2.65 |
| Example VI | 85 | 1.1 | 3.3 | 2.96 |
| Example VII | 86 | 1.2 | 3.8 | 3.23 |
| Example VIII | 85 | 1.1 | 3.6 | 3.19 |
| Example IX | 81 | 1.2 | 3.7 | 3.04 |

Contact angle measurements were performed on dry films of cements with a Contact Angle Measuring System G10 (Kruss). The surface free energy was calculated by the approach introduced by Fowkes, in which the total surface tension is considered as a sum of independent terms, each representing a particular intermolecular force and by the application of the equation of Owens and Wendt, which is an extension to a so-called “polar” component. The liquids used for this purpose were methylene iodide and distilled water.

Wetability of the modified cements was studied due to its importance on the interactions with biological species. Values of contact angle for the cements prepared with DMAO together with the values of the surface energy of solid, Υₛ, and those of dispersive, Υₛ^{d}, and polar, Υₛ^{p}, components are given in Table 9 below. The cement formulated with 5.84 wt % DMAO content showed an increase in hydrophobicity of the surface as reflected by the significant decrease (p<0.001) on the contact angle with methylene iodide. This increase arises from the presence of the fatty acid residues present in the activator molecules. However, total surface free energy did not appreciably change due to the compensation of both polar and dispersive components.

TABLE 9

| | Activator | | θ | Υₛ | | |
|---|---|---|---|---|---|---|
| EX- | Concentration | θ | methylene | (mN/ | Υₛ^{d} | Υₛ^{p} |
| AMPLE | (wt-%) | water | iodide | m) | (mN/m) | (mN/m) |
| Control | 1.9 | 77 | 40 | 44.2 | 39 | 4.2 |
| | (DMT) | [1.8] | [3.3] | | | |
| Example | 5.84 | 79 | 33 | 46.8 | 43 | 3.6 |
| II | (DMAO) | [2.6] | [1.8] | | | |
| Example | 5.84 | 79 | 34 | 45.5 | 43 | 3.2 |
| III | (DMAO) | [1.1] | [1.6] | | | |
| Example | 2.57 | 78 | 40 | 44.1 | 39 | 4.0 |
| IV | (DMAO) | [2.3] | [2.4] | | | |

Thus, for the bone cement precursor compositions of the present invention, the examples show that the polymerisation exotherm was at least 20° C. lower than commercial formulations and the extent of polymerisation monitored by the amount of residual monomer was comparable to standard formulations. The resultant bone cements also exhibited good mechanical properties.

1. A composition, for use in a dental or bone cement precursor composition, which comprises one or more compounds of the general structure: 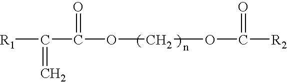
wherein:
R₁ is H, methyl, ethyl, propyl or isopropyl,
n=1, 2, 3 or 4,
R₂ is a side chain having the general structure shown below:
H₃C—(CH₂)ₐ—(CH(OH)CH₂)_{b}—(CH═CHCH₂)_{c}—(CH═CH(CH₂)₇)_{d}—
wherein:
a=4 to 16
b=0 or 1
c=0 or 1
d=0 or 1
and wherein the total number of carbon atoms in the side chain R₂ is not greater than 17 and one or more free radical activators.

2. A composition as claimed in claim 1 wherein R₂ in the compound of Formula (I) is the side chain of a fatty acid.

3. A composition as claimed in claim 2 wherein R₂ in the compound of Formula (I) is CH₃(CH₂)₁₀, CH₃(CH₂)₁₄, CH₃(CH₂)₁₆, CH₃(CH₂)₇CH═CH(CH₂)₇, CH₃(CH₂)₅CH(OH)CH₂CH═CH(CH₂)₇ or CH₃(CH₂)₄CH═CHCH₂CH═CH(CH₂)₇.

4. A composition as claimed in claim 1 wherein R₂ in the compound of Formula (I) is unsaturated.

5. A composition as claimed in any one of the preceding claims when the compound of Formula (I) is included therein in an amount of from 2 to 30 wt %, more preferably from 5 to 15 wt %, based on the total weight of the composition.

6. A composition as claimed in any one of the preceding claims which also includes methylmethacrylate therein in an amount of from 70 to 98 wt %, more preferably from 85 to 95 wt %, based on the total weight of the composition.

7. A composition as claimed in any one of the preceding claims which comprises one or more tertiary amine activators in an amount of up to 7 wt %, based on the total weight of the composition, and/or one or more inhibitors in an amount of up to 0.01 wt % and/or one or more stabilisers in an amount of up to 0.01 wt %.

8. A composition as claimed in claim 7 wherein the tertiary amine activator is from N,N-diethyl-p-toluidine, dimethylamino ethylmethacrylate or N,N-dimethylaniline.

9. A composition as claimed in claim 7 wherein the tertiary amine activator is a compound of the general structure: 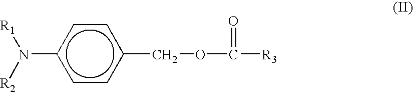
wherein:
R₁ is methyl, ethyl, propyl, isopropyl, butyl, isobutyl or tert-butyl,
R₂ is methyl, ethyl, propyl, isopropyl, butyl, isobutyl or tert-butyl,
R₃ is a side chain having the general structure shown below:
H₃C—(CH₂)ₐ—(CH(OH)CH₂)_{b}—(CH═CHCH₂)_{c}—(CH═CH(CH₂)₇)_{d}—
wherein:
a=4 to 16
b=0 or 1
c=0 or 1
d=0 or 1
and wherein the total number of carbon atoms in the side chain R₃ is not greater than 17.

10. A composition as claimed in claim 7 wherein the inhibitor and/or stabiliser is a quinone based compound.

11. A composition, for use as a bone cement precursor composition, which comprises a solid phase and a liquid phase, wherein the liquid phase comprises a composition as claimed in any one of the preceding claims.

12. A compound of the general structure: 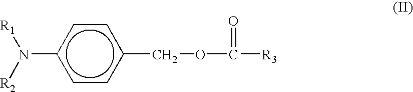
wherein:
R₁ is methyl, ethyl, propyl, isopropyl, butyl, isobutyl or tert-butyl,
R₂ is methyl, ethyl, propyl, isopropyl, butyl, isobutyl or tert-butyl,
R₃ is a side chain having the general structure shown below:
H₃C—(CH₂)ₐ—(CH(OH)CH₂)_{b}—(CH═CHCH₂)_{c}—(CH═CH(CH₂)₇)_{d}—
wherein:
a=4 to 16
b=0 or 1
c=0 or 1
d=0 or 1
and wherein the total number of carbon atoms in the side chain R₃ is not greater than 17,provided that other said compound is not 4-N,N-dimethyl-amino benzyl laurate.

13. A compound as claimed in claim 12 wherein R₃ is a side chain of a fatty acid.

14. A compound as claimed in claim 12 or claim 13 wherein R₃ is a side chain selected from CH₃(CH₂)₁₀, CH₃(CH₂)₁₄, CH₃(CH₂)₁₆, CH₃(CH₂)₇CH═CH (CH₂)₇, CH₃(CH₂)₅CH(OH)CH₂CH═CH(CH₂)₇ or CH₃(CH₂)₄CH═CHCH₂CH═CH(CH₂)₇.

15. A composition, for use in a dental or bone cement precursor composition, which comprises one or more compounds of Formula (II) as claimed in any one of claims 12 to 14 and one or more polymerisable monomers.

16. A composition as claimed in claim 15 which comprises a compound of Formula (II) in a amount of up to 7 wt %, more preferably in the range of from 3 to 7 wt %, based on the total weight of the composition.

17. A composition as claimed in claim 15 or claim 16 which also includes methylmethacrylate in an amount of from 70 to 95 wt %, more preferably from 85 to 90 wt %, based on the total weight of the composition.

18. A composition as claimed in any one of claims 15 to 17 which comprises one or more compounds of Formula (I) as defined in claim 1 in an amount of from 2 to 30 wt %, more preferably from 5 to 15 wt %, based on the total weight of the composition.

19. A composition as claimed in any one of claims 15 to 18 which comprises one or more inhibitors in an amount of up to 0.01 wt %, and/or one or more stabilisers in an amount of up to 0.01 wt %.

20. A composition as claimed in claim 19 wherein the inhibitor and/or stabiliser is a quinone based compounds.

21. A composition, for use as a dental or bone cement precursor composition, which comprises a solid phase and a liquid phase wherein the liquid phase, comprises a composition as claimed in any one of claims 15 to 20.

22. A composition, as claimed in claim 11 or claim 21 wherein the solid phase comprises beads of prepolymerised MMA or copolymers of MMA with other monomers, said beads being present in an amount of from 70 to 95 wt %, preferably from 80 to 95 wt %, based on the total weight of the solid phase.

23. A precursor composition as claimed in any one of claims 11, 21 or 22 wherein the solid phase comprises one or more initiators in an amount of up to 3 wt % and/or one or more radiopaque agents in an amount of up to 20 wt %, based on the total weight of the solid phase.

24. A composition as claimed in claim 23 wherein the initiator is benzoyl peroxide and/or the one or more radiopaque agents is/are selected from barium sulphate or zirconia.

25. A dental or bone cement which has been produced by curing a Composition as claimed in any one of claims 11 and 21 to 24.

26. A process for forming a cement which comprises providing a solid phase and a liquid phase, mixing the two phases together and allowing polymerisation to occur, wherein the solid phase comprises polymer beads and one or more radical initiators and the liquid phase is a composition as claimed in any one of claims 1 to 8 or 15 to 20.
